(19) United States
(12) Patent Application Publication
(10) Pub. No.: US 2005/0033429 A1
(43) Pub. Date: Feb. 10, 2005

(54) Spinal expansion apparatus

(76) Inventor: Robert W. H. Kuo, Taipei (TW)

Correspondence Address: Troxell Law Office PLLC;Suite 1404, 5205 Leesburg Pike, Falls Church, VA, 22041, US

(21) Appl. No.: 10/633,491

(22) Filed: Aug. 5, 2003

(51) Int. Cl. A61F 2/44

(52) U.S. Cl. 623/17.11

(57) ABSTRACT

A spinal expanding apparatus includes multiple supports extendable out of passages in a body and a bolt movably received in the body. The bolt has multiple grooves in an outer periphery of the bolt to correspond to bottom conical heads of the supports. Therefore, advancement of the bolt is able to force the supports to simultaneously extend out of the passages of the body. Each of the supports has a travelling distance the same as the others so that two adjacent spines are evenly separated.

### BACKGROUND OF THE INVENTION

1. Field of the Invention

The present invention relates to an expansion apparatus, and more particularly to a spinal expansion apparatus which is able to securely and easily sandwiched between two adjacent spinal disks to rectify the deformed spine.

2. Description of Related Art

With reference to FIGS. 7 and 8, a conventional spinal expansion device is composed of a primary sleeve (50) with an inner threading (51) and a secondary sleeve (60) with an outer threading (61) corresponding to the inner threading (51) of the primary sleeve (50).

When the conventional spinal expansion device is in application, a tool (70) is inserted into the secondary sleeve (60) to rotate the secondary sleeve (60) inside the primary sleeve (50). Due to the mutual mating between the inner threading (51) and the outer threading (61), the secondary sleeve (60) is advancing in the primary sleeve (50). Further, because the secondary sleeve (60) has a conical cross section, the advancement of the secondary sleeve (60) in the primary sleeve (50) forces the primary sleeve (50) to expand in size. Thus, after the primary sleeve (50) is expanded, the original deformed two adjacent spinal disks are able to be forced to move further apart from each other such that the distance between the two adjacent spinal disks is maintained normal.

However, when the secondary sleeve (60) is advancing in the primary sleeve (50), the expansion of the primary sleeve (50) due to the conical cross section of the secondary sleeve (60) is not evenly distributed. That is, the distance between two adjacent spines is not evenly expanded, which may cause a serious consequence.

To overcome the shortcomings, the present invention tends to provide an improved spinal expansion apparatus to mitigate the aforementioned problems.

### SUMMARY OF THE INVENTION

The primary objective of the present invention is to provide an improved spinal expansion apparatus having two rows of supports oppositely and movably received in the body. When the two rows of supports are forced to extend out of the body, each row of the supports engage with a spine to evenly separate two adjacent spines.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of the spinal expansion apparatus of the present invention;

FIG. 2 is a schematic cross sectional view showing the internal structure of the body of the spinal expansion apparatus of the present invention;

FIG. 3 is a schematic view showing that when the support is not in use;

FIG. 4 is a schematic view showing that the support is retracted in the passage in the body when the support is in engagement with a spine;

FIG. 5 is a schematic view before the spinal expansion apparatus is inserted into two adjacent spines;

FIG. 6 is a schematic view showing the status after the spinal expansion apparatus is inserted into two adjacent spines and the supports are activated;

FIG. 7 is an exploded perspective view of a conventional spinal expansion device; and

FIG. 8 is a schematic view showing the application of the conventional spinal expansion device in FIG. 7.

### DETAILED DESCRIPTION OF THE PREFERRED EMBODIMENT

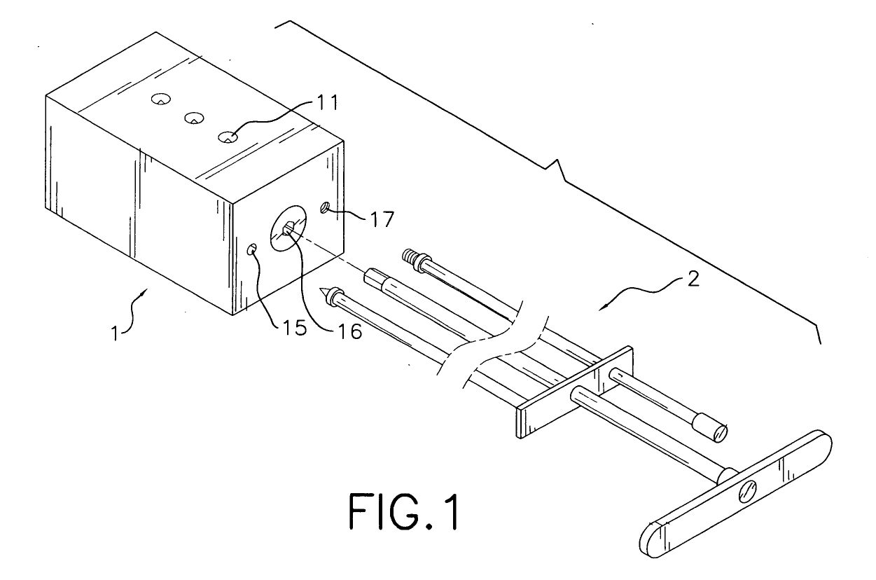

With reference to FIG. 1, the spinal expansion apparatus in accordance with the present invention includes a body (1) and a tool (2).

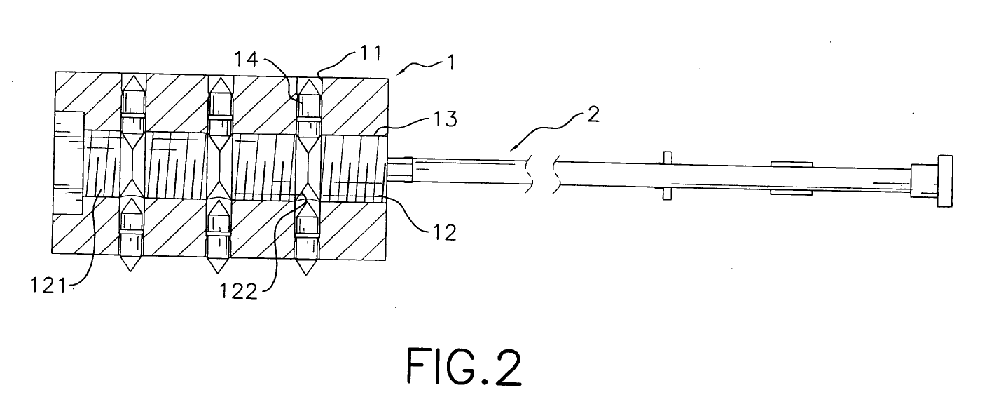

With reference to FIG. 2 and still taking FIG. 1 for reference, the body (1) has passages (11) oppositely defined in the body (1), a bolt (12) movably received in a channel (13) in the body (1) to orthogonally communicate with the passages (11) and supports (14) each movably received in a corresponding one of the passages (11). The bolt (12) has an outer threading (121) formed on an outer periphery of the bolt (12) and V-shaped grooves (122) defined in the outer periphery of the bolt (12) to correspond to the supports (14).

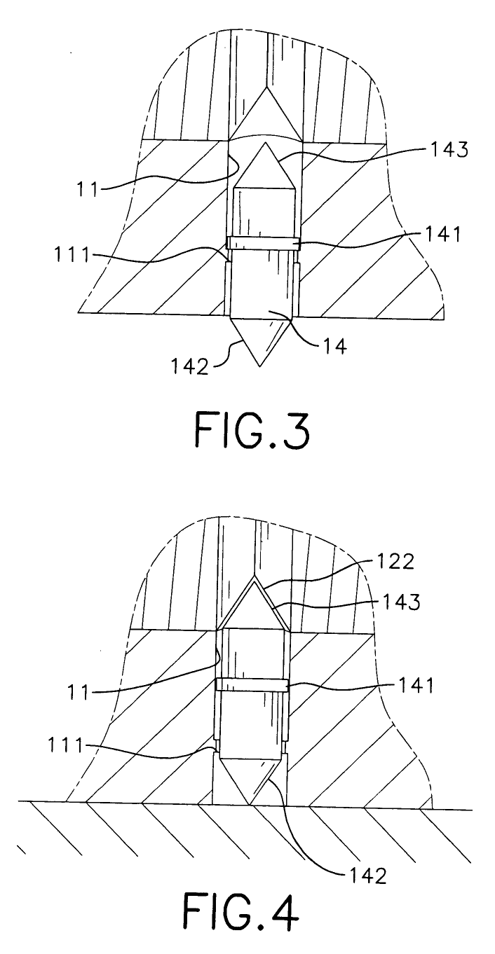

With reference to FIGS. 3 and 4, it is noted that a shoulder (111) is formed in each of the passages (11) and each support (14) has a flange (141) formed on an outer periphery of the support (14) to correspond to the shoulder (111) so that the support (14) is maintained in the corresponding passage (11) of the body (1). Furthermore, each of the supports (14) has a top conical head (142) and a bottom conical head (143) oppositely formed relative to the top conical head (142). The bottom conical head (143) is engaged with a bottom face defining the V-shaped groove (122) and the top conical head (142) is facing outside the passage (11). From the depiction in FIGS. 3 and 4, it is noted that while the support (14) is not in use, the support (14) is supported by the interaction between the flange (141) and the shoulder (111) and the top conical head (142) extends out of the passage (11). Meanwhile, the bottom conical head (143) is not in engagement with the bottom face defining the V-shaped groove (122).

However, when the top conical head (142) is engaged with a surface, e.g. the surface of a spine, the support (14) is forced into the passage (11) and the bottom conical head (142) is engaged with the bottom face defining the V-shaped groove (122).

Still further, with reference to FIG. 1 again, the body (1) has a hole (15), a driving hole (16) and a threaded hole (17) all defined in a side face of the body (1). The tool (2) includes a stabilizing bar (21), a driving bar (22) and a securing bar (23) with a threaded head (231) formed on the front portion of the securing bar (23). A connection plate (24) is provided to securely engage with a distal end of the stabilizing bar (21). Both the driving bar (22) and the securing bar (23) rotatably extend through the connection plate (24) and the driving bar (22) has a handle (221) fixedly provided on a distal end of the driving bar (22). That is, the first distal end of the stabilizing bar (21) is securely and fixedly connected to the connection plate (24), the first distal end of the driving bar (22) is securely connected to the handle (221) and the first distal end of the securing bar (23) extends through the connection plate (24).

The stabilizing bar (21) has a sharp second distal end corresponding to the hole (15). The driving bar (22) has a second distal end so configured to mate with the driving hole (16) and threaded head (231) of the securing bar (23) corresponds to the threaded hole (17).

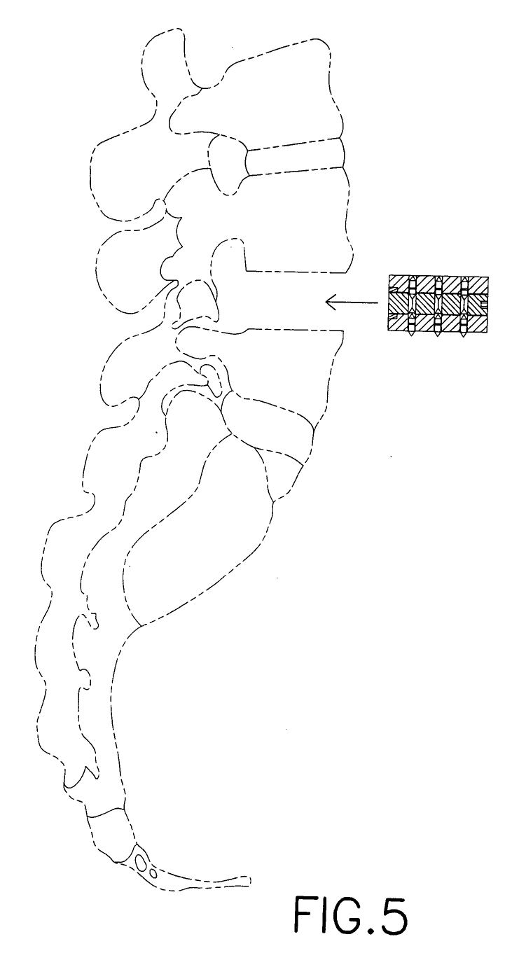

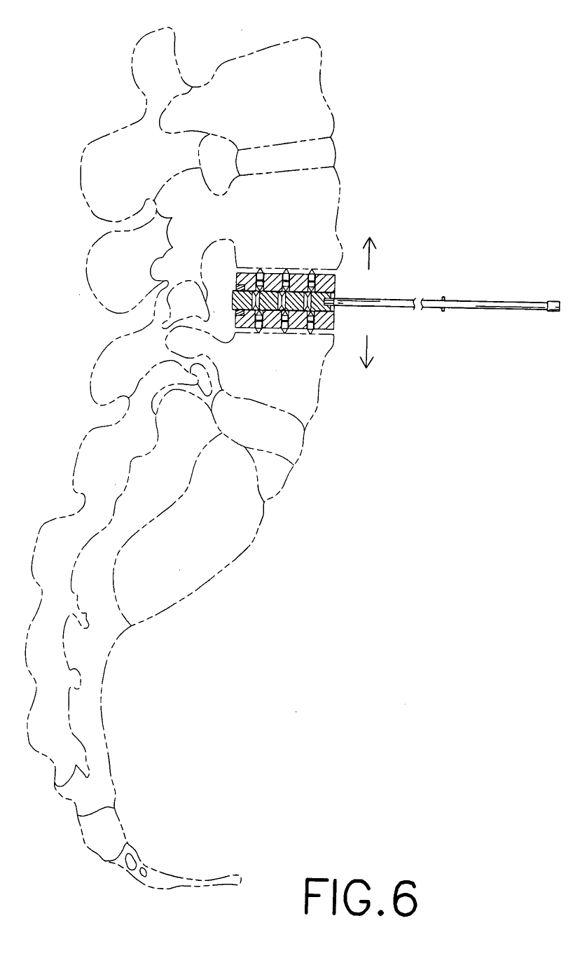

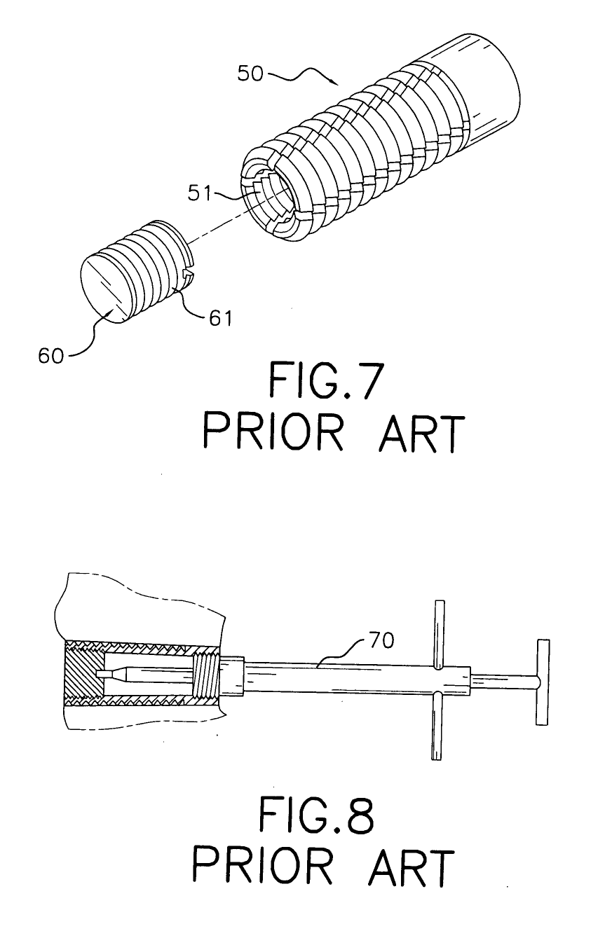

With reference to FIGS. 5 and 6, when the apparatus of the present invention is in application, the operator first inserts the body (1) into two adjacent spines (shown in dashed lines). Then, the operator extends the stabilizing bar (21) into the hole (15) to stabilize the tool (2) relative to the body (1). The driving bar (22) is then extended into the driving hole (16) and the securing bar (23) is screwed into the threaded hole (17) to secure the connection between the tool (2) and the body (1).

Thereafter, the operator rotates the handle (221) to further extend the bolt (12) in the channel (13). When the bolt (12) is advancing in the channel (13), side faces of the V-shaped grooves (122) abut the bottom conical heads (143) of the supports (14) and eventually force the supports (14) to extend from the passages (11). Because the bolt (12) is cylindrical, the advancement of the supports (14) out of the passages (11) is simultaneous and thus the travelling distance of each of the supports (14) is the same as the others. Therefore, the distance between two adjacent spines is evenly expanded.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A spinal expansion apparatus comprising:
a body having a channel defined inside the body to movably receive therein a bolt and passages defined to respectively and movably receive therein a support, wherein the passages are orthogonal to the channel; and
means for advancing the bolt inside the channel; and
means for extending the supports out of the passages,
whereby the supports are simultaneously extending out of the passages and travel a same distance resulting from the advancement of the bolt and thus two adjacent spines are respectively and evenly separated.

2. The spinal expansion apparatus as claimed in claim 1, wherein the bolt has V-shaped grooves respectively defined in an outer periphery of the bolt to correspond to one of the supports.

3. The spinal expansion apparatus as claimed in claim 1, wherein each of the supports has a bottom conical head facing the bolt and a top conical head facing out of the passage for engagement with a surface of the spine.

4. The spinal expansion apparatus as claimed in claim 2, wherein each of the supports has a bottom conical head facing the bolt to engage with a bottom face defining the V-shaped groove and a top conical head facing out of the passage for engagement with the surface of the spine.

5. The spinal expansion apparatus as claimed in claim 1, wherein each of the passages has a shoulder formed in an inner periphery defining the passage and each of the supports has a flange formed on an outer periphery of the support to prevent the support from falling out of the passage.

6. The spinal expansion apparatus as claimed in claim 3, wherein each of the passages has a shoulder formed in an inner periphery defining the passage and each of the supports has a flange formed on an outer periphery of the support to prevent the support from falling out of the passage.

7. The spinal expansion apparatus as claimed in claim 4, wherein each of the passages has a shoulder formed in an inner periphery defining the passage and each of the supports has a flange formed on an outer periphery of the support to prevent the support from falling out of the passage.

8. The spinal expansion apparatus as claimed in claim 1, wherein the bolt advancing means includes a driving hole defined in a side face of the body and a driving bar with a first distal end so configured that when the first distal end of the driving bar is inserted into the driving hole, rotation of the driving bar is able to force the bolt to move in the channel.

9. The spinal expansion apparatus as claimed in claim 8, wherein the bolt advancing means further includes a hole defined in the side face of the body and a stabilizing bar connected to the driving bar via a connection plate and having a first distal end detachably received in the hole.

10. The spinal expansion apparatus as claimed in claim 9, wherein the bolt advancing means further includes a threaded hole defined in the side face of the body and a securing bar connected to the driving bar via the connection plate and having a threaded distal end screwed into the threaded hole.

11. The spinal expansion apparatus as claimed in claim 7, wherein the bolt advancing means includes a driving hole defined in a side face of the body and a driving bar with a first distal end so configured that when the first distal end of the driving bar is inserted into the driving hole, rotation of the driving bar is able to force the bolt to move in the channel.

12. The spinal expansion apparatus as claimed in claim 11, wherein the bolt advancing means further includes a hole defined in the side face of the body and a stabilizing bar connected to the driving bar via a connection plate and having a first distal end detachably received in the hole.

13. The spinal expansion apparatus as claimed in claim 12, wherein the bolt advancing means further includes a threaded hole defined in the side face of the body and a securing bar connected to the driving bar via the connection plate and having a threaded distal end screwed into the threaded hole.

14. The spinal expansion apparatus as claimed in claim 1, wherein the support extending means includes V-shaped grooves defined in an outer periphery of the bolt and bottom conical heads each formed on a distal end of the supports to be alternatively engage with a bottom face defining the V-shaped groove so that when the bolt is moving in the channel, the abutment between the bottom faces defining the V-shaped grooves and the bottom conical heads of the supports is able to force the supports to extend out of the passages.

15. The spinal expansion apparatus as claimed in claim 7, wherein the support extending means includes V-shaped grooves defined in an outer periphery of the bolt and bottom conical heads each formed on a distal end of the supports to be alternatively engage with a bottom face defining the V-shaped groove so that when the bolt is moving in the channel, the abutment between the bottom faces defining the V-shaped grooves and the bottom conical heads of the supports is able to force the supports to extend out of the passages.

16. The spinal expansion apparatus as claimed in claim 8, wherein the support extending means includes V-shaped grooves defined in an outer periphery of the bolt and bottom conical heads each formed on a distal end of the supports to be alternatively engage with a bottom face defining the V-shaped groove so that when the bolt is moving in the channel, the abutment between the bottom faces defining the V-shaped grooves and the bottom conical heads of the supports is able to force the supports to extend out of the passages.

17. The spinal expansion apparatus as claimed in claim 9, wherein the support extending means includes V-shaped grooves defined in an outer periphery of the bolt and bottom conical heads each formed on a distal end of the supports to be alternatively engage with a bottom face defining the V-shaped groove so that when the bolt is moving in the channel, the abutment between the bottom faces defining the V-shaped grooves and the bottom conical heads of the supports is able to force the supports to extend out of the passages.

18. The spinal expansion apparatus as claimed in claim 10, wherein the support extending means includes V-shaped grooves defined in an outer periphery of the bolt and bottom conical heads each formed on a distal end of the supports to be alternatively engage with a bottom face defining the V-shaped groove so that when the bolt is moving in the channel, the abutment between the bottom faces defining the V-shaped grooves and the bottom conical heads of the supports is able to force the supports to extend out of the passages.

19. The spinal expansion apparatus as claimed in claim 13, wherein the support extending means includes V-shaped grooves defined in an outer periphery of the bolt and bottom conical heads each formed on a distal end of the supports to be alternatively engage with a bottom face defining the V-shaped groove so that when the bolt is moving in the channel, the abutment between the bottom faces defining the V-shaped grooves and the bottom conical heads of the supports is able to force the supports to extend out of the passages.
